(19) United States
(12) Patent Application Publication
(10) Pub. No.: US 2009/0292560 A1
(43) Pub. Date: Nov. 26, 2009

(54) Disease Management Interface System

(76) Inventor: Paul Woods, Hibbing, MN (US)

Correspondence Address: PATENT DEPARTMENT;LARKIN, HOFFMAN, DALY & LINDGREN, LTD., 1500 WELLS FARGO PLAZA, 7900 XERXES AVENUE SOUTH, BLOOMINGTON, MN, 55431, US

(21) Appl. No.: 12/254,370

(22) Filed: Oct. 20, 2008

Related U.S. Application Data
(60) Provisional application No. 60/965,235, filed on Oct. 18, 2007.

(51) Int. Cl. G06Q 50/00; A61B 5/00

(52) U.S. Cl. 705/3; 705/2; 600/300

(57) ABSTRACT

A disease management interface system to improve the management of chronic diseases, disorders and conditions, comprising: by linking evidence-based protocols and desired management outcomes to a particular patients data through the use of rules-based decision engines, and presentation of the results of these decisions at opportune points in the process of care for a patient. Simplified presentation of information is unique and promotes appropriate management decisions, and protocol driven ordering programming supports reliability and safety of care. The use of a complete patient-specific cluster of process and outcome measures, rather than evaluation of the process and outcome measures for specific diseases considered in isolation of co-existing conditions, brings a patient centered focus to the management of these conditions. The disease management interface system is independent of a specific proprietary electronic health record, but versions will be programmed for use with all electronic health records.

### RELATED APPLICATIONS

This application claims the benefit of U.S. Provisional application 60/695,235 entitled “Disease Management Interface System”, filed on Oct. 18, 2007.

### BACKGROUND

The present invention generally relates to medical records management. More specifically, the present invention provides a tool for health care facilities to provide improved management of chronic disease.

The challenges of chronic disease in United States health care are placing increasing burden on healthcare providers and provider organizations to deliver evidence-based care to the population bearing these disease states and conditions. The complexity of these conditions, economic constraints produced by declining reimbursement, the public reporting of outcomes, and decreasing numbers of providers combine to make it necessary to consider safer, more comprehensive, and efficient ways to manage these patients.

To provide effective disease management, care systems and care providers must have access to information which will help to manage the care strategy followed and the care provided. To be effective, manageable amounts of information must be provided in an easy to read format. Further, the information should also help give the care provider the most current practices related to the applicable disease.

Any patient contact with a provider system is an opportunity to improve the care of their chronic disease. Frequently patients call for purposes other than care of their chronic disease, from prescription refills to lab results or general inquiries. Non-clinical staff such as schedulers and other ancillary staff need to be alerted to overdue tests and procedures without having to search for these, and to use the opportunity this contact affords to easily schedule those overdue tests and procedures.

Data must inform care. Irrelevant data should not be provided. This is the principle of converting data in to information. Parsimonious provision of relevant data reduces information overload and improves the likelihood that the information will be used by the provider. Current decision support applications are characterized by the presentation of an overwhelming abundance of data, which requires the provider to sift through things to extract information that can be used to provide care.

In a more efficient system, the information should be presented without the provider having to search for it. With an aging population and an increasing prevalence of obesity (and further complications such as Diabetes Mellitus), chronic disease increasingly consumes more health care financial and non-financial resources. Accordingly, as chronic disease management increases in importance to provider systems, every interaction with the provider organization should be used as an opportunity to improve the care for patients with chronic disease. As mentioned above, current decision support applications require a provider to search numerous locations in the health record to find data and perform more manual functions to compare them to longitudinal trends. This effectively “penalizes” a provider who attempts to provide care based on a complete picture of a patient's data.

The shortest path a provider can take should be the best care practice. Current electronic health record and decision support tools are designed in a way that ordering tests and procedures takes more time than not doing it. This places a premium on not doing what should be done and again penalizes a provider who is attempting to provide the best care. Intelligently designed systems will do the opposite by shortening the path to optimum care.

Targets and goals of therapy from the medical evidence base literature change at a rapid pace. Due to the fast moving changes and updates to this information it is often difficult for providers to keep up. Presentation of a patient's management data should be provided in an intuitive fashion including the standard level of care that a provider organization is using as a target. This must be presented in a way that integrates the “standard” with the patient's data. Most current presentations of care standards are presently separately from the patient's data in a non-intuitive form. As can be imaged, this created an additional step which is easily omitted by those involved. Most providers never bother to look for what the “Best Practice” is, and if presented it usually dismiss it without action.

The information must take in to account and integrate the care for all chronic disease processes present in a particular patient. Most patients suffering from a chronic disease have one or more other diseases (co-morbidities) which interact with each other in some manner. This might include different management outcomes targets (blood pressure targets in patients with chronic kidney disease, diabetes, and high blood pressure are all different). Recognition of the presence of these co-morbidities may influence a practitioner's choice of medication, dosing changes, or even decision whether to treat an abnormality at all. Current electronic health records and decision support tools treat each individual chronic disease as if it existed in that patient in isolation of all co-morbidities. A useful decision support tool should integrate care for the patient for all chronic disease and resolve differing management targets.

In addition to the above discussed issues related to disease management, the information presented by a system should also help give the care provider the most current practices related to the applicable disease. This information is available in the literature, but is not appropriately linked to patient care interface products to be effectively used.

### SUMMARY

The disease management interface system of the present invention leverages the capabilities of information systems and electronic health records to assist providers in improving the quality and reliability of care for patients with chronic conditions, while also decreasing the time and effort spent doing so.

In one embodiment, the disease management interface system is a software application that uses automated evidence-based decisions and protocols and presents them, as well as all relevant management data at every appropriate opportunity to optimize the processes of care and outcomes for patients with chronic diseases. This is accomplished by accessing the patient's data contained in the electronic health record, and using this data along with evidence-based standards to generate computer screen interfaces that provide appropriate opportunities for scheduling of tests and procedures that are out of date. Additionally, management outcomes are provided in conjunction with comparisons to evidence-based standards to provide success measures. Additionally, longitudinal management trends are presented to the provider during an encounter that permits timely and comprehensive management of all the chronic disease processes for each patient.

To provide a more effective tool, one embodiment of the disease management system interface presents information to the appropriate system user in a manner to most effectively manage the relevant task to be undertaken. For example, when the care system is utilizing the system, a scheduling interface is provided which concisely presents information related to necessary procedures to be scheduled. In a similar manner, when the system is accessed by a care provider, information related to the treatment strategy is provided, thus allowing the provider to easily assess the patient's condition and determine the best next steps. In this embodiment, the two interfaces are specifically configured to provide meaningful and relevant information, while also providing the mechanisms to access additional data if necessary. An additional feature that could be added to this embodiment provides the ability to easily access and present history information which can comprehensively illustrate trends during disease management.

### BRIEF DESCRIPTION OF DRAWING

Further objects and advantages of the present invention can be seen from reading the following detailed description of the preferred embodiments, in conjunction with the drawings in which:

FIG. 1 is a conceptual representation of the visible embodiments of the DMIS as represented by two example screenshots for a fictitious patient;

FIG. 2 is a representation of the unpopulated unseen conceptual matrix structure used in generating the scheduling matrix interface;

FIG. 3 is a representation of an example populated conceptual matrix structure used in generating the scheduling matrix interface;

FIG. 4 is a representation of the scheduling matrix interface seen by a member of the provider system having contact with an example fictitious patient;

FIG. 5 is a representation of the unpopulated unseen conceptual matrix structure used to generate the management matrix interface;

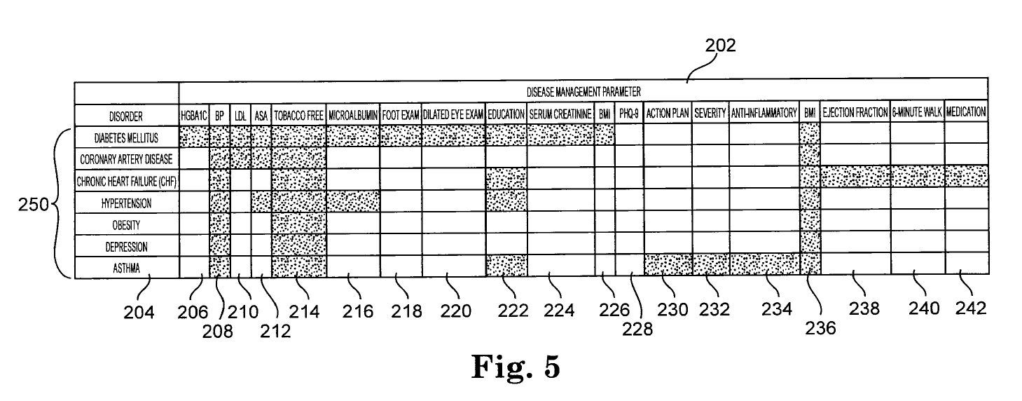

FIG. 6 is a representation of an example populated conceptual matrix structure used to generate the management matrix interface;

FIG. 7 is a representation of the management matrix interface seen by a provider having contact with an example fictitious;

FIG. 8 is a representation of the quick flow chart interface presenting longitudinal trends for the patient in FIG. 7; and

FIG. 9 is a schematic diagram of an exemplary networked system capable of supporting the disease management interface system.

### DETAILED DESCRIPTION OF THE PREFERRED EMBODIMENTS

The disease management interface system is designed around several overarching principles. The first is automation to improve reliability, standardize care, and reduce workload. The second is simplification—that the shortest path to delivery of care should be the best process of care. The third is maximization of qualification and licensure—that work should be done by those at the maximum extent of their licensure and education. The final principle is efficiency—that rework should be minimized or eliminated.

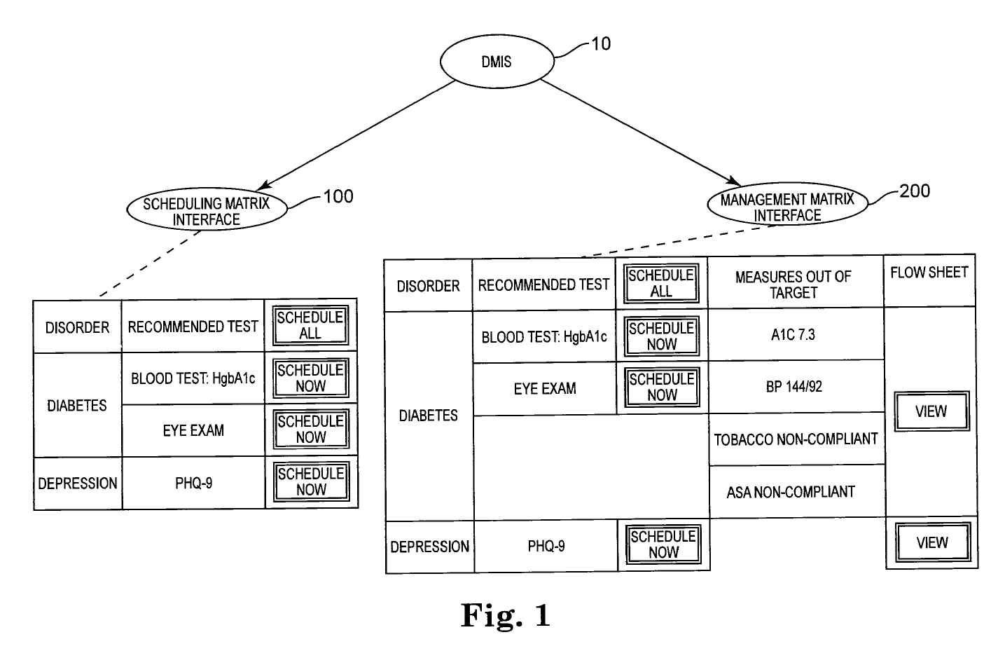

In a preferred embodiment, the disease management interface system 10 generates two interfaces that are presented to the care team. Referring to FIG. 1, a scheduling matrix interface 100 is presented to clinical and non-clinical staff whenever the patient makes contact with the health care providing organization. This will include schedulers, clinical assistants, nurse-on-line, pharmacy refill teams, and others that the patient may make contact with. The management matrix interface 200 is generated and presented to the provider team, which may consist of physicians, mid-level providers, nurse clinicians, and others involved in management of the patient's chronic disease.

The management and clinical course of a chronic disease is complex. The medical literature has generated and evidence base that allows quantification of the quality of care a patient with chronic disease receives. This is separated in to two categories. Process measures indicate whether a procedure or test was performed at the right time to adequately manage the disease. Optimization of process measures is accomplished by the scheduling matrix interface 100. Outcome measures are indicators of whether the disease process was managed to the desired clinical endpoints as defined by the body of medical literature. Optimization of outcome measures is accomplished by the management matrix interface FIG. 1 sets forth a conceptual separation of these two concepts as generally incorporated in the various embodiments of the invention.

Process measures are derived from consensus evidence-based guidelines. There is little or no variation among providers or applicable patients. Ordering of these tests and procedures can be done by simple rules-based decision algorithms. Clinical preparedness and judgment are not required, and thus these can be done by non-clinical staff under these decision algorithms. The disease management interface system links a rules-based decision engine to patient-specific data contained in defined fields in the electronic health record to accomplish this.

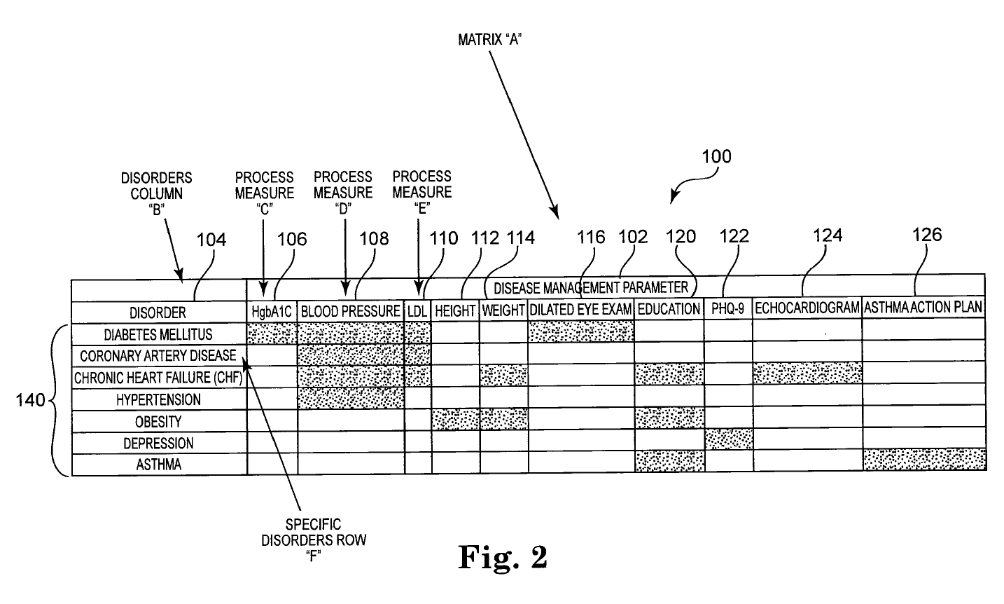

As illustrated in FIG. 2, the software of one embodiment maintains a conceptual matrix structure 102 to organize and ultimately present a scheduling matrix interface 100, The conceptual disorder and process measure matrix 102 contains a disorder column 104 which lists the chronic condition the health care organization wishes to manage with the software. The remainder of the columns 106-126, etc. contain the process measures associated with the management of the chronic conditions contained in disorder row 104. While this matrix 102 is shown conceptually in FIG. 2, it is not actually visible to the user of the software. Rows (disorders) 140 and columns (process measures), e.g. 106-126, can be added or removed as desired by the organization using the system.

Each field in the conceptual matrix 102 contains an individual rules-based decision engine. These are programs which use simple rules to determine if a patient's process measures are within guidelines. The guidelines against which the patient's data are compared are determined by the user organization's quality or chronic disease management departments. The rules-based decision engines are part of the system and/or software package.

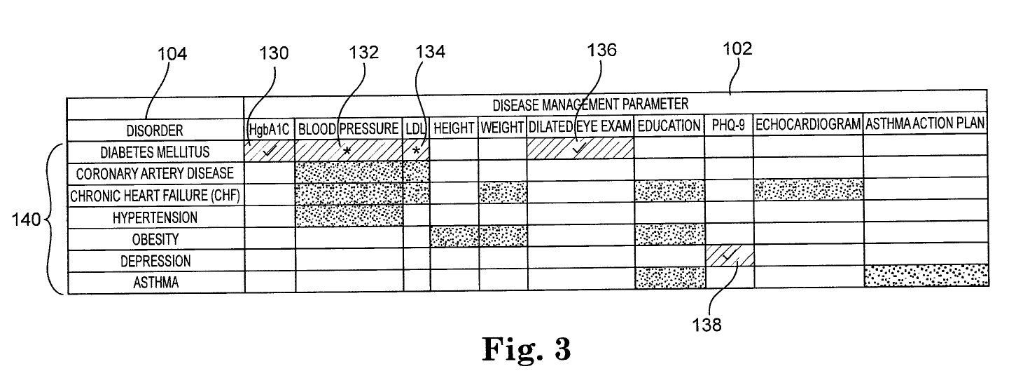

When activated, the software takes the patient identifiers (name, date of birth, medical record number) and accesses his/her electronic health record. ICD codes in that patient's problem list are compared to the disorders in the first column of the matrix. If there are any matches, the row for all the disorders applicable for that patient become active. Each cell of the row, containing its own decision engine, generates a decision of whether a related process is overdue. The matrix then becomes populated with fields either overdue or not. As illustrated, FIG. 3 appropriate indicators 130-138 are placed in the appropriate cells. In this example, fields associated with this patient's diabetes and depression are marked. Here, indicators are used to identify overdue parameters (x) and up to date parameters (√).

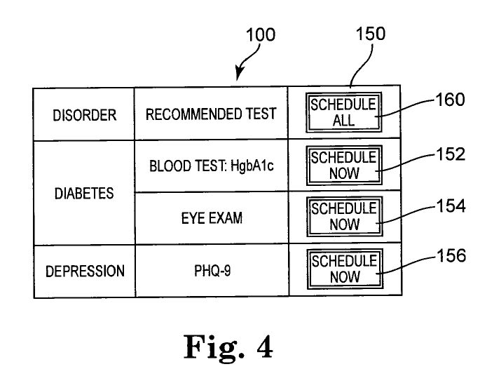

After populating the fields of the matrix, the matrix is then collapsed to only disorders and process measures that are overdue. This step is best achieved by identifying the indicators existing in appropriate cells. This leaves a patient-specific composite matrix 128 of disorders carried by that patient which contain overdue process measures (see FIG. 4).

FIG. 4 illustrates the form and content of the scheduling matrix interface 100, which is presented to clinical and non-clinical staff. The right hand column 150 has fields which contain programming which allows scheduling of the overdue measure by a single corresponding button 152, 154, 156. There is also a master button 160 which schedules all overdue tests, shortening time expenditure required to comply with evidence-based guidelines.

The scheduling matrix interface 100 will be presented to clinical and non-clinical staff with every contact the patient makes to the health care providing organization.

Enhancing compliance with evidence-based guidelines for outcomes measures requires clinical knowledge and judgment that limits the utility of simple rules alone. Nonetheless, several of the same principles can be used to present outcomes in a way to provide decision support for the provider team. These include simplification of the ordering processes and presentation of overdue measures. When presenting information related to treatment outcomes, only measures which are out of the desired range should be presented in this interface. Absent is a management outcome indicator to the provider that shows a measure is up to date and within the desired range. This eliminates clutter from the interface associated with too many measures. The management matrix interface 200 should be presented to the provider team with every contact a patient has with the health care provider team whether it is relevant to the patient's chronic conditions or not.

The information presented in this interface (termed the management matrix interface) has several components. It contains the current management outcomes which are outside the desired range. It also gives the standard that the patient should be treated to, Additionally, an indication of the linear management trends of the disease process for that individual patient can easily be presented. This gives the provider a snapshot of where the state of the patient's diseases are, where they have been, where they are going, and where they should be in a simple screen interface.

A similar conceptual matrix 202 to that used to generate the scheduling matrix interface 100 is used in the management matrix interface 200. This conceptual matrix 202 is also not seen by the user. The first column 204 contains the same chronic conditions and diseases (disorders) used by the first matrix. Similarly, related parameters are listed in corresponding columns 206-242. The rows 250 contain rules-based decision engines that compare management outcomes accessed in the patient's electronic health record with desired outcomes. New management parameters can be added at the health care organization's discretions as a new column.

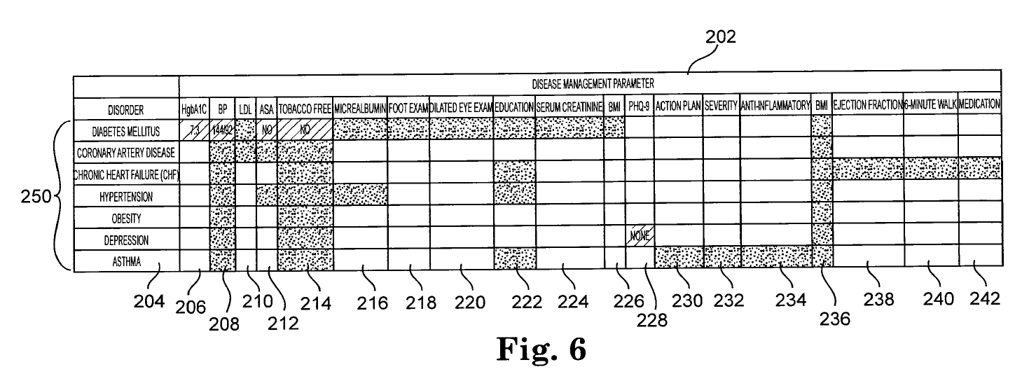

Field population in the matrix is done by comparing the patient's outcomes with the desired outcomes for that disorder. FIG. 6 illustrates one example of this where cells corresponding to a patient's diabetes and depression are populated. The rules-based decision engine then generates a decision of “in desired range”, “outside desired range and stable or improving”, and “outside desired range and worsening.” As shown in FIG. 6 these various comparisons are illustrated with highlighting or appropriate shading.

It can be seen that there may be multiple different outcomes for a parameter that is important for multiple disorders, such as blood pressure. Rather than having the presentation of multiple blood pressures, the management matrix interface software decision engine evaluates the patient's outcome down each column and compares the patient's result to the highest standard for all disorders borne by that patient. This will then determine if the patient's result is “out of range.” Although he or she may meet the management standard for some of the disorders, he or she should be treated to the highest standard for all the disorders present. This then truly becomes a patient-specific, rather than disease-specific management tool.

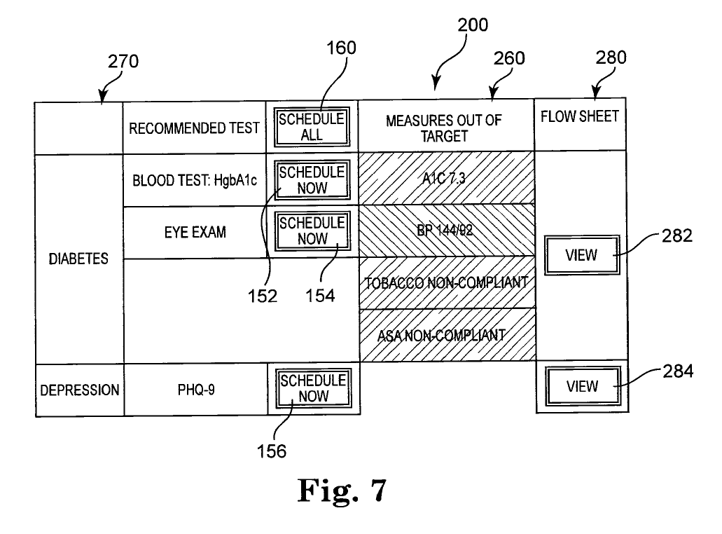

After populating the fields of the matrix 202, it is then collapsed to only disorders and management measures that are outside the desired range. This leaves a patient-specific composite matrix of disorders carried by that patient which contain management measures which are not optimal (see FIG. 7).

The second column of the management matrix interface 200 contains the same process measure decision results contained in the scheduling matrix interface, as it is likely that many patients will get to the provider team without having their process measures scheduled at another point in the care process. Including this component in the management matrix interface allows the provider team a clear summary of overdue measures and enables them to order the tests quickly using the schedule buttons. The fourth column 260 (Measures out of Target) is generated by the collapse of the invisible matrix illustrated in FIG. 6. This gives a patient-specific summary of the management status of all the chronic conditions borne by that patient.

Three features of this summary column are noted in keeping with the principles of design. First, measures which are up to date and in the target range are not presented (removing the need to recall what the current management standard is). Second, the current “out of target” management parameters are presented. Finally, color-coding indicates the linear trend of the patient's condition. Yellow coding indicates that the “out of target” parameter is stable or improving. Red coding indicates that the parameter is worsening.

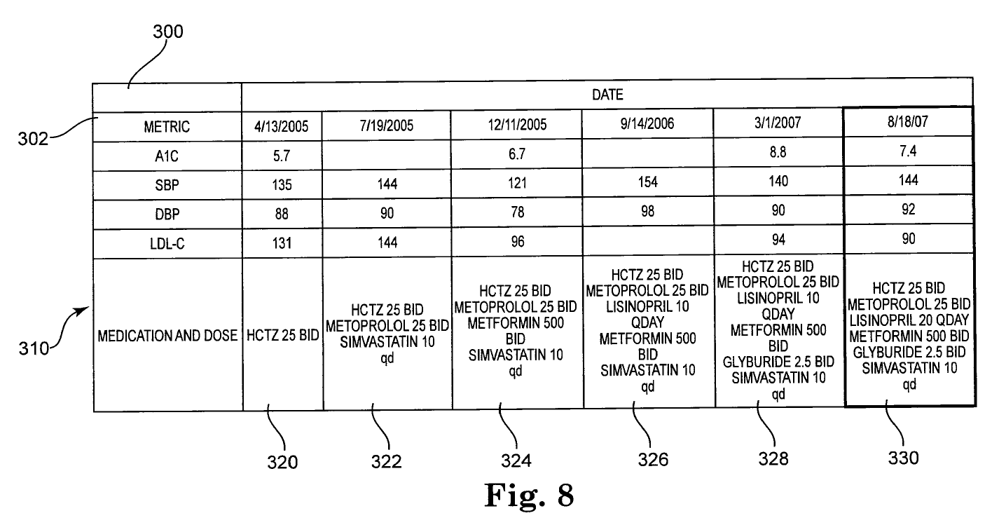

The fifth column 280 of the management matrix interface allows the creation of a quick management flow chart 300 as more fully shown in FIG. 8. In this particular illustration, a first view button 282 and a second view button 284 are illustrated. Each of these view buttons (282 and 284) are available to provide more detailed information related to the disorders listed in disorder column 270. Again, this example illustrates Diabetes and Depression as the two listed disorders. Further, first view button 282 corresponds to the Diabetes disorder while second view button 284 corresponds to the depression disorder.

Referring now to FIG. 8, an exemplary quick flow chart 300 is illustrated. This quick flow chart 300 will only be generated for the parameters which are currently out of the target range. The out of range information is listed in the final/most recent column 330 and maintains the same indications listed in the interface of FIG. 7. This flow chart 300 will present a comparison of target or standards data to the current outcomes over time, and present the medications related to management of these disorders as the results for a particular disorder are evaluated. More specifically, a number of columns 320-330 are presented, corresponding to dates when relevant data was obtained. The quick flow chart includes a list 310 at the bottom of each column that contains all the medications as of the date indicated related to the disorders contained in the flow chart. As the cursor is hovered on a data point (for instance a particular Hgb A1C 302) the software brings forward (enlarges and bolds) medications related to that particular disorder, allowing the provider to examine quickly the response to management changes that have been made.

The result of using the disease management interface system is a patient-specific approach to managing all of the chronic disorders that an individual may possess. It promotes proper ordering of tests and procedures which are overdue at an access point when they are not currently done, with a minimum expenditure of time as well as the safety assured by evidence-based protocols and logical application through computer algorithms. As well, management outcomes are presented to provider teams at every point of contact a patient with chronic diseases and disorders have, and those outcomes are presented in a way that enhance decision-making and promote active management.

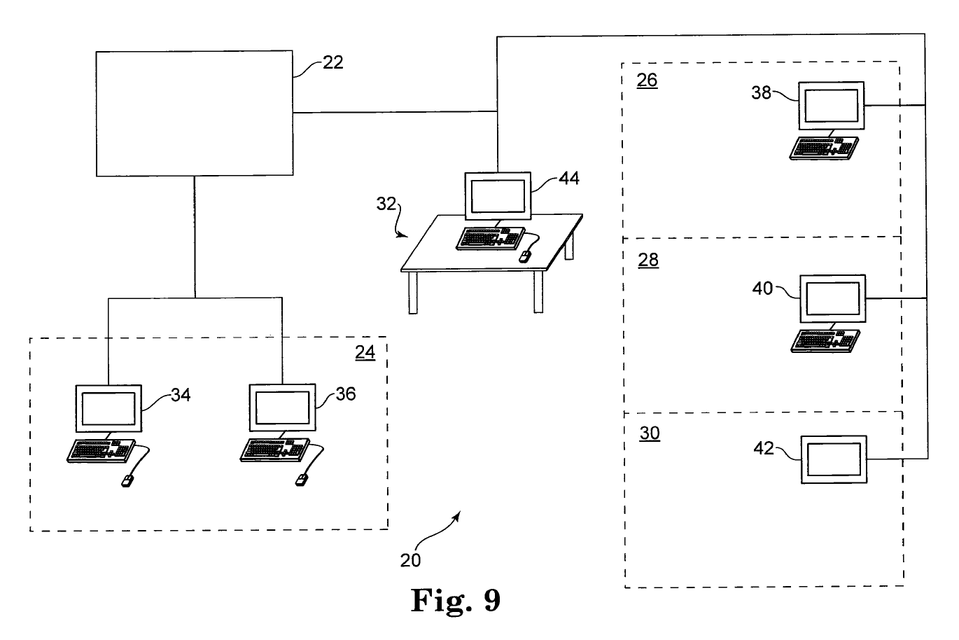

As can be anticipated, the above discussed interface can be utilized on several different systems. For example, FIG. 9 schematically illustrates one potential system which could exist in various health care facilities. In this illustration, an information management system 20 includes several components conveniently positioned for use by the appropriate individuals involved. System 20 includes a central records system 22 which maintains patient records for the patients supported by the health care organization. The illustrated health care organization includes an administrative portion 24, a number of examining rooms 26, 28, 30, and a nurse station 32. Within the administrative portion of the organization are two terminals 34 and 36. It is contemplated that these terminals could be utilized by receptionists, schedulers, or records managers. Similarly, examining room terminals 38, 40 and 42 are located in the first examining room 26, the second examining room 28 and the third examining room 30, respectively. Lastly, a nurse station terminal 44 is positioned at the nurses station 32. Each of the above listed terminals may take on many forms, which may include standard computer terminal, touch screen devices, portable devices, mini-computers, laptop computers, etc. In each case, these devices will interface with the central records system 22 in order to proved the interface utilities that are discussed above.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited in the particular embodiments which have been described in detail therein. Rather, reference should be made to the appended claims as indicative of the scope and content of the present invention.

What is claimed is:

1. A disease management interface system to improve the management of chronic diseases, disorders and conditions, comprising:
access to a database containing patient records for a population of patients served by a provider organization,
a processor for poling the database in response to a user inquiry to obtain data related to a patient including an exiting care strategy and a listing of care data, and linking evidence-based protocols and desired management outcomes to the particular patients data through the use of rules-based decision engines, and
a display generator to present the results of these decisions at a predetermined points in the process of care for a patient, the display generator generating a scheduling interface when the system is accessed by a care system thus allowing the user to easily schedule any outstanding procedures typical for the existing care strategy and a management interface when accessed by the care giver to allow the care giver to easily assess the success of the care strategy.

2. The disease management interface system of claim 1 wherein the scheduling interface and the management interface are both presented in a grid format, wherein the scheduling interface presents outstanding procedures necessary for the care strategy in a graphical format with the condition listed in one portion of the matrix, at least one procedure listed in one portion of the matrix and completion status listed in an intersecting portion of the matrix, and wherein the success of the care strategy is presented graphically with the condition listed in one portion of the matrix, a most recent assessment is listed in another portion, a target is listed in yet another portion, and results are listed in the intersecting portions of the matrix.

3. A method for effectively presenting information to health care providers and health care systems to efficiently manage chronic disease, the method comprising:
establishing contact with a records management system whenever contact is made with a relevant patient by a health care system to poll data related to any existing chronic diseases for the relevant patient and to identify any outstanding procedures related to any existing chronic diseases;
presenting to the health care system a listing of the outstanding procedures and presenting a scheduling mechanism to allow the health care system to quickly schedule procedures through a presented interface, wherein the presented interface lists the existing chronic condition along with the outstanding procedures in an interrelated format and the scheduling mechanism provides a connection to a scheduling utility;
establishing a connection with the health records system whenever contact is made with the relevant patient by a health care provider to poll and retrieve the data related to any existing chronic disease of the relevant patient, the second interface further comparing the retrieved data with established standards of care to determine if the retrieved data is in compliance with the standards or out of compliance; and
presenting any retrieved data that is out of compliance for further analysis and assessment, wherein the out of compliance data is presented in a matrix format with the out of compliance measures being highlighted.

4. The method of claim 3 further comprising the presentation of a expansion tool which allows the presentation of a quick flow chart listing the history of the out of compliance readings.

5. The method of claim 3 further comprising the presentation of a schedule all tool when the outstanding procedures are listed.

6. The method of claim 3 wherein the determination of compliance related to standards involves the retrieval of standards information from a standards database and the comparison of measured values with the corresponding values contained in the standards database, the matrix format presentation including a listing of the related chronic condition along with a listing of the corresponding out of compliance measured value and the highlighting achieved using a predetermined color coding scheme.

7. The method of claim 6 wherein the matrix further comprises the presentation of a expansion tool which generates a quick flow chart listing the history of the out of compliance readings.
